(19) United States
(12) Patent Application Publication
(10) Pub. No.: US 2023/0034559 A1
(43) Pub. Date: Feb. 2, 2023

(54) AUTOMATED PREDICTION OF CLINICAL TRIAL OUTCOME

(71) Applicant: Sunstella Technology Corporation, Las Vegas, NV (US)

(72) Inventors: Tianfan Fu, Atlanta, GA (US); Kexin Huang, San Mateo, CA (US); Jimeng Sun, Las Vegas, NV (US)

(21) Appl. No.: 17/749,065

(22) Filed: May 19, 2022

Related U.S. Application Data
(60) Provisional application No. 63/223,029, filed on Jul. 18, 2021.

(51) Int. Cl. G16H 50/50; G16C 20/70; G16C 20/50; G16B 15/30

(52) U.S. Cl. CPC G16H 50/50; G16C 20/70; G16C 20/50; G16B 15/30

(57) ABSTRACT

A system for prediction of clinical trial outcome. The system includes: a processor of a trial prediction (TP) node connected to at least one cloud server node over a network configured to host a machine learning (ML) module; a memory on which are stored machine-readable instructions that when executed by the processor, cause the processor to: receive a clinical trial (CT) data, parse the CT data to derive drug molecules data, disease information data, and trial protocols data, encode the drug molecules data, the disease information data, and the trial protocols data into corresponding embeddings, generate knowledge pre-trained embeddings using external knowledge data, and provide the knowledge pre-trained embeddings to the ML module for prediction of the CT outcome.

### RELATED APPLICATION

Under provisions of 35 U.S.C. § 119(e), the Applicant claims benefit of U.S. Provisional Application No. 63/223,029 filed on Jul. 18, 2021, and having inventors in common, which is incorporated herein by reference in its entirety.

It is intended that the referenced application may be applicable to the concepts and embodiments disclosed herein, even if such concepts and embodiments are disclosed in the referenced application with different limitations and configurations and described using different examples and terminology.

### FIELD OF DISCLOSURE

The present disclosure generally relates to clinical trials, particularly to an intelligent AI-based automated system for the prediction of a clinical trial outcome.

### BACKGROUND

Clinical trials are research studies involving trial participants to evaluate the safety and efficacy of medical devices and drugs that have been newly developed to treat diseases and health conditions. Clinical trials are typically conducted after the medical device or drug has been tested on animals. Clinical trials typically produce the evidence upon which governmental regulatory agencies rely when approving a medical device or drug for human use.

Clinical trials follow strict scientific standards in order to produce reliable results. Clinical trials are crucial for drug development but are time-consuming, expensive, and often burdensome on patients. More importantly, clinical trials face uncertain outcomes due to issues with efficacy, safety, or problems with patient recruitment. Clinical trials are an indispensable step towards developing a new drug, where human participants are tested in responding to a treatment (e.g., a drug or drug combinations) for treating target diseases. The costs of conducting clinical trials are extremely expensive (up to hundreds of millions of dollars) and the time required to run a clinical trial is very long with low success probability. However, many factors such as the inefficacy of the drug, drug safety issues, and poor trial protocol design can cause the failure of a clinical trial. If a reliable prediction of a clinical trial success probability could be made, the trials that would inevitably fail with their current design can be modified or redesigned prior to implementation of the clinical trial.

Recently limited attempts of trying to predict individual components in clinical trials have been made. Specifically, leverage gradient-boosted decision trees have been used to predict the effect of antidepressant treatment in improving depressive symptoms based on electroencephalographic (EEG) measures. Some conventional solutions use an ensemble classifier based on weighted least squares support vector regression to predict the clinical trial outcome based on drug-property and target-property features. Also, the recurrent neural network has been used to predict the phase-3 trial results based on phase-2 results in terms of longitudinal data. Some solutions expand beyond optimizing individual components to predict trial outcomes for 15 disease groups based on disease-only features using linear models, including random forest and logistic regression.

Despite these initial efforts, several following limitations impede the utility of existing trial outcome prediction models. The first limitation of the conventional solutions is a limited task scope. Existing solutions either focus on predicting individual components of trials such as patient-trial matching or only covering disease groups of which the disease-specific features are available. Although these solutions are potentially helpful for a limited part of the trial design, they do not answer the fundamental problem—will this trial be approved, or what probability of the trial approval may be expected. The second limitation of the conventional solutions is the limited features used for prediction. The existing solutions only leverage restricted disease-specific features, which cannot be generalized to other diseases. These solutions largely ignore the multi-faceted risks, including drug safety, treatment efficiency, and trial recruitment, where abundant information is publicly available for assessing those different risks. For example, the biomedical knowledge base provides explicit biochemical structures of the drug molecules, and previous trial history for similar diseases can be useful for trial outcome prediction.

Yet another limitation of the existing solutions is handling complex relations among trial components and outcomes. The existing works are often over-simplified, which significantly impedes them from modeling the complicated relations among various trial components.

Accordingly, a system and method for an intelligent AI-based automated prediction of a clinical trial outcome is desired.

### BRIEF OVERVIEW

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

One embodiment of the present disclosure provides a system for automated prediction of a clinical trial outcome. The system includes: a processor of a trial prediction (TP) node connected to at least one cloud server node over a network configured to host a machine learning (ML) module; a memory on which are stored machine-readable instructions that when executed by the processor, cause the processor to: receive a clinical trial (CT) data, parse the CT data to derive drug molecules data, disease information data, and trial protocols data, encode the drug molecules data, the disease information data, and the trial protocols data into corresponding embeddings, generate knowledge pre-trained embeddings using external knowledge data, and provide the knowledge pre-trained embeddings to the ML module for prediction of the CT outcome.

Another embodiment of the present disclosure provides a method that includes one or more of: receiving a clinical trial (CT) data; parsing the CT data to derive drug molecules data, disease information data, and trial protocols data; encoding the drug molecules data, the disease information data, and the trial protocols data into corresponding embeddings; generating knowledge pre-trained embeddings using external knowledge data; and providing the knowledge pre-trained embeddings to an ML module for prediction of the CT outcome.

Another embodiment of the present disclosure provides a computer-readable medium including instructions for receiving a clinical trial (CT) data; parsing the CT data to derive drug molecules data, disease information data, and trial protocols data; encoding the drug molecules data, the disease information data, and the trial protocols data into corresponding embeddings; generating knowledge pre-trained embeddings using external knowledge data; and providing the knowledge pre-trained embeddings to an ML module for prediction of the CT outcome.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 illustrates a network diagram of a system for an intelligent AI-based automated prediction of a clinical trial outcome consistent with the present disclosure;

FIG. 2 illustrates a network diagram of a system including detailed features of a trial prediction (TP) server node consistent with the present disclosure;

FIG. 3A illustrates a flowchart of a method for an intelligent AI-based automated prediction of a clinical trial outcome consistent with the present disclosure;

FIG. 3B illustrates a further flow chart of a method for knowledge driven predictive model for trial outcome prediction consistent with the present disclosure;

FIG. 4 illustrates a diagram of input data embedding process consistent with the present disclosure;

FIG. 5 illustrates a diagram of drug embedding and missing drug imputation for clinical trial outcome prediction consistent with the present disclosure;

FIG. 6 illustrates a further diagram of drug property embedding consistent with the present disclosure;

FIG. 7 illustrates a diagram of disease embedding for clinical trial outcome prediction consistent with the present disclosure;

FIG. 8 illustrates a diagram of connection of different nodes used for node embedding for the trial outcome prediction interaction graph consistent with the present disclosure;

FIG. 9 illustrates a diagram for generation of a reasoning matrix associated with predicted trial outcome consistent with the present disclosure;

FIG. 10 illustrates a diagram for determining a trial outcome probability based on trial embedding consistent with the present disclosure; and

FIG. 11 illustrates a block diagram of a system including a computing device for performing the method of FIGS. 3A and 3B.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of processing job applicants, embodiments of the present disclosure are not limited to use only in this context.

The present disclosure provides a system, method and computer-readable medium for an intelligent AI-based automated prediction of a clinical trial outcome.

Historical clinical trial data, and massive knowledge base about approved and failed drugs may bring a new opportunity for using machine learning AI-based models to tackle the key question—can a clinical trial success probability be accurately predicted. The disclosed embodiments may address these challenges by formulating an all-cause and an all-phase clinical trial outcome prediction task, which has practical utility for a clinical trial design. In one embodiment, a Hierarchical Interaction NeTwork (HINT) that explicitly simulates each clinical trial component and the complicated relations among them is provided. The HINT uses a graph neural network-based approach to model the interplay among various trial risks, including drug safety, treatment efficiency, and trial recruitment. It also covers a wide range of drugs and indications (e.g., diseases to be treated). A disclosed model framework can generalize over new trials given the easily accessible drug, disease and protocol information.

According to the disclosed embodiments, problem formulation may be implemented as follows. A clinical trial aims to validate the safety and efficacy of a treatment set towards a target disease set on a patient group.

Definition 1 (Treatment Set). In a trial, the treatment set includes one or multiple drug candidates, denoted by M={m₁, . . . , m_{N}_{ₘ}}. The system of the disclosed embodiment is restricted to trials that aim at discovering new indications of drug candidates. Other trials that involve surgeries or devices are not considered. Drug molecule is used.

Definition 2 (Target Disease Set). Each trial targets one or more diseases, denoted D={d₁, . . . , d_{N}_{_{d}}}, dᵢ represents the disease code of the i-th disease. An ICD-10 code is used based on the 10th revision of the International Statistical Classification of Diseases 402.

Definition 3 (Trial Protocol). Each clinical trial protocol consists of inclusion and exclusion criteria in a text format, describing the characteristics of desired patients for the trial, such as age, gender, medical history, target disease conditions, and current health status. It is denoted as C=[c₁, . . . , c_{N}_{ₚ}].

Problem 1 (Clinical Trial Outcome Prediction). The trial outcome is a binary label y, y=1 indicates trial success (i.e., the trial received approval status) and 0 indicates failure (i.e., all the other status). In this trial outcome prediction task, the system trains a deep neural network y=f_{θ}(M,D,C) to predict the outcome, based the model parameters θ.

In one embodiment, an input embedding module encodes multi-modal data, including drug molecules, disease information, and trial protocols to embeddings. Next, these embeddings are fed into a knowledge embedding module to generate knowledge embeddings pre-trained using external knowledge including drug pharmaco-kinetics and disease risk data. Then, the interaction graph module connects all the embeddings via domain knowledge to fully capture various trial components and their complex relations as well as their influences on trial outcomes. Based on that, the HINT learns (i.e., trains) dynamic attentive graph neural network to predict trial outcome.

An input embedding module may learn embedding from drug molecules; disease information; trial protocols.

The drug molecules are represented by molecule graphs. The disclosed embodiments may use message passing network (MPN) to encode molecular graphs and average over embeddings of all the molecules in the treatment set as the treatment embedding:

hₘ=mean([MPN(m₁), . . . ,MPN(m_{N}_{ₘ})])∈R^{d},

where M={m₁, . . . ,m_{N}_{ₘ}} is the drug treatment set.

Disease information also affects the outcome. For example, drugs in oncology have much lower approval rates than ones in infectious diseases. The disease information comes from its description and its corresponding ontology, such as disease hierarchies like International Classification of Diseases (ICD). D={d₁, . . . , d_{N}_{_{d}}}(Definition 2), the diseases in the trial are represented as:

h_{d}=mean([GRAM(dᵢ), . . . ,GRAM(d_{N}_{_{d}})])∈R^{d},

where GRAM (dᵢ) represents embedding of dᵢ using GRAM (graph-based attention model), which leverages the hierarchical information inherent to medical ontologies.

The trial protocol describes eligibility criteria, i.e., patient recruitment requirements. Each inclusion or exclusion criteria corresponds to a sentence. Clinical-BERT may be applied to get the sentence representation and average over all the sentences:

hₚ=mean([ClinicalBERT(c₁), . . . ,ClinicalBERT(c_{N}_{ₚ})])∈R^{d}.

One special challenge associated with clinical trial data is that there can be missing data on molecular information due to proprietary information. This poses a problem since representation of many nodes depends on the molecular information. It has been observed that there exists high correlation between the drug molecule, disease and protocol features.

One special challenge associated with clinical trial data is that there can be missing data on molecular information due to proprietary information. This poses a problem since representation of many nodes depends on the molecular information. It has been observed that there exists high correlation between the drug molecule, disease and protocol features. Thus, the disclosed embodiments provide a missing data imputation module based on learning embeddings that capture inter-modal correlations and intra-modal distribution. In particular, the imputation module IMP(⋅) uses disease and protocol embedding (h_{d}, hₚ) to recover molecular embedding hₘ:

[symbol]

=IMP(h_{d},hₚ)

Here, the system adopts MSE (Mean Square Error) loss as the learning objective to minimize the distance between the ground truth molecule embedding and predicted one.

In one embodiment, the HINT utilizes external knowledge to pretrain knowledge nodes and further enhance those input embeddings. In case of pharmaco-kinetics knowledge, the embeddings are pre-trained using the pharmaco-kinetics (PK) knowledge about how the body reacts to the intake of a drug. The success of a clinical trial highly depends on factors including drugs' pharmaco-kinetics properties and disease risk. Specifically, the HINT system may leverage various public PK experimental scores. Based on this information, the system may pre-train on prediction models for Absorption, Distribution, Metabolism, Excretion, Toxicity (ADMET) properties, which are used to observe how a drug interacts with the body. Absorption describes how drugs travels from administration site to the site of action. Distribution measures the drug's ability to move through the bloodstream. Metabolism measures the duration of a drug's efficacy. Excretion measures how much of drug's toxic components can be removed from the body. Toxicity model measures damage to the body. For each pharmaco-kinetics property, binary labels may be used to indicate whether the drug is desirable in this property. Concretely, for each *∈{A,D,M,E,T}, the system pre-trains a knowledge node based on the label y_{*}∈{0,1} as follows:

h_{*}=X_{*}(hₘ)∈R^{d}, y_{*}=Sigmoid(FC(h_{*})), min−y_{*} log ŷ_{*}−(1−y_{*})log(1−y_{*}),

where X_{*} is a two-layer highway network. FC is a fully-connected layer. Binary cross-entropy is a loss criteria. After training, the drug in each trial can be fed to the pre-trained model to obtain absorption/distribution/metabolism/excretion/toxicity embeddings.

With respect to a disease risk, the disclosed embodiments consider the knowledge distilled from historical trials of the target diseases. The historical trial success rate may be used for the disease. As detailed statistics for trial success rate of each disease at different trial phases are widely available, the system considers that as the supervision signal to train the disease risk prediction model:

h_{R}=R(h_{d}), ŷ_{R}=Sigmoid(FC(h_{R})), min−y_{R} log [symbol] −(1−y_{R})log(1− [symbol] ),

where h_{d} is the disease embedding defined above, h_{R} is disease risk embedding, y_{R}∈{0,1} is the risk label. Cross-entropy loss is minimized. After training, the disease information in the trial is fed to the pretrained model to obtain disease risk embedding.

In one embodiment, a hierarchical interaction graph G may be constructed to connect input data sources and important factors affecting clinical trial outcomes. The graph reflects the real-world trial development process and consists of four tiers of nodes that are connected between tiers:

Input nodes include drugs, diseases and protocols with node features of input embedding hₘ,h_{d},hₚ∈R^{d}.

External knowledge nodes include ADMET embeddings h_{A},h_{D},h_{M},h_{E},h_{T}∈R^{d} and disease risk embedding h_{R}∈R^{d}. These representations are pretrained on external knowledge.

Aggregation nodes include:

(a) Interaction node h_{I} connecting disease h_{d}, drug molecules hₘ and protocols hₚ;

(b) Pharmaco-Kinetics node h_{PK} connecting ADMET embeddings:

h_{A},h_{D},h_{M},h_{E},h_{T}∈R^{d};

(c) augmented interaction node h_{V} that augments interaction node h_{I} using disease risk node h_{R}.

Prediction node: h_{pred} connects pharmaco-kinetics node h_{PK} and augmented interaction node h_{V} to make the prediction.

According to the disclosed embodiments, the aggregation nodes are implemented as follows. The PK (Pharmaco-Kinetics) node is to gather information about ADMET properties. PK (Pharmaco-Kinetics) embedding is obtained by:

h_{PK}=f_{K}([h_{A},h_{D},h_{M},h_{E},h_{T}]^{T})∈R^{d}

where f_{K}(⋅) a one-layer fully-connected layer followed by a two-layer highway network.

Then, the system models the interaction among drug molecule, disease and protocol, the interaction node embedding is:

h_{I}=f_{I}([hₘ,h_{d},hₚ]^{T})∈R^{d},

f_{I}(⋅) has the same architecture as f_{K}(⋅).

The Augmented interaction node combines (i) trial risk of the target disease h_{R} and (ii) the interaction node h_{I}:

h_{V}=f_{V}([h_{R},h_{I}]^{T})∈R^{d},

where f_{V} uses the same architecture as f_{K}(⋅), f_{I}(⋅).

The Prediction node summarizes pharmaco-kinetics and augmented interaction

h_{pred}=fₚ([h_{PK},h_{V}]^{T})∈R^{d},

where fₚ uses the same architecture as f_{I}, fᵥ.

The Dynamic Attentive Graph Neural Network is implemented as follows. The trial embeddings provide initial representations of different trial components and their interactions via a graph. To further enhance interaction, a dynamic attentive graph neural network is designed. Mathematically, in interaction graph G, nodes are trial components, edges are the relations among them.

A∈{0,1}′ denotes the adjacency matrix of G, K=13 is the node number. Node embeddings H⁽⁰⁾ are initialized by stacking representation of all the components:

H⁽⁰⁾=[hₘ,h_{d},hₚ,h_{A},h_{D},h_{M},h_{E},h_{T},h_{R},h_{PK},h_{V},h_{pred}]^{T}∈R^{K×d}

The disclosed embodiment, advantageously, enhances the interaction between nodes using graph convolutional network (GCN). The updating rule for the l-th layer is:

H^{(l)}=RELU(B^{(l)}+(V⊙A)H^{(l-1)}W^{(l)}), l=1, . . . ,L,

where L is GCN depth. In the l-th layer, H^{(l)}∈R^{K×d} is node embeddings, B^{(l)}, W^{(l)} are the bias/weight parameter, ⊙ represents element-wise multiplication. Different from conventional GCN, the system employs a dynamic layer-independent attentive matrix V E R₊^{K×K}, V_{i,j} which measures the importance of the edge between i-th and j-th nodes. It is parameterized by a two-layer fully-connected neural network with ReLU and Sigmoid activation function in the hidden and output layer:

V_{i,j}=f_{V}([hᵢ,hⱼ]^{T})∈R₊.

V is the reasoning matrix where each entry corresponds to the reasoning score among each trial component. It is then element-wisely multiplied to adjacency matrix A so that it assigns weights to edges in interaction graph G.

The prediction and training. After GNN message-passing, updated representations for trial components are obtained. To generate the trial success prediction 9, the last-layer (L-th) representation of the prediction node is fed into a one-layer fully-connected network with sigmoid function, and leverage binary cross-entropy loss to guide training:

ŷ=Sigmoid(FC(h_{pred}^{(L)})), min−y log ŷ−(1−y)log(1−9),

where y∈{0,1} is the ground truth. The HINT is trained in an end-to-end manner.

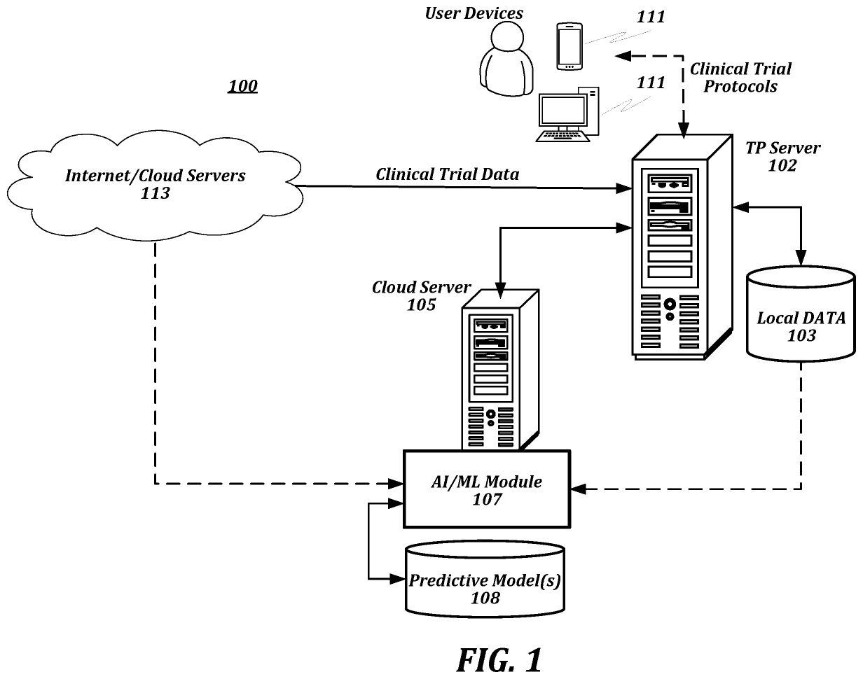

FIG. 1 illustrates a network diagram of a system for an intelligent AI-based automated determination of clinical trial outcome consistent with the present disclosure.

Referring to FIG. 1, the example network 100 includes the trial prediction (TP) server node 102 connected to a cloud server node(s) 105 and servers 113 over a network. The cloud server node(s) 105 is configured to host an AI/ML module 107. The TP server node 102 may receive clinical trial parameters (e.g., drug molecules, disease information, and trial protocols data) from a user device 111 that may be a smartphone, a tablet, a laptop/PC, etc. The TP server node 102 may acquire pharmaco-kinetics data from the servers 113. As discussed above, the TP server node 102 may query available pharmacological databases (not shown) based on the clinical trial parameters and may store the drug molecules data, pharmaco-kinetics data and disease information data in a local database 103. The TP server 102 may predict a trial outcome by ingesting feature vectors data derived from the drug molecules data, disease information, and trial protocols data into an AI/ML module 107.

The AI/ML module 107 may generate a predictive model(s) 108 to predict patient having the disease the drug based on data collected and stored in the local database 103. Once the trial outcome is positively predicted a list of the potential participants is established based on the trial protocols data, these participants may be contacted and invited to the clinical trial.

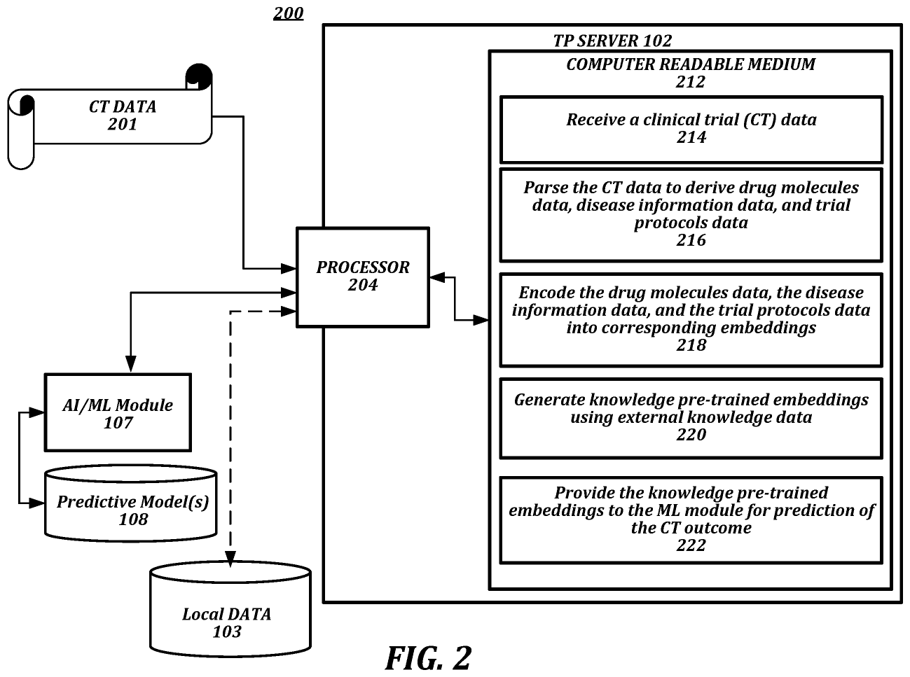

FIG. 2 illustrates a network diagram of a system including detailed features of a trial prediction (TP) server node 102 consistent with the present disclosure.

Referring to FIG. 2, the example network 200 includes the TP server node 102 connected to a cloud server node(s) 105 over a network. The cloud server node(s) 105 is configured to host an AI/ML module 107. As discussed above with reference to FIG. 1, the TP server node 102 may receive clinical trial data 201 including drug molecules, disease information, and trial protocols data.

The AI/ML module 107 may generate a predictive model(s) 108 based on historical trial data provided by the TP server 102 from a local data storage. As discussed above, the AI/ML module 107 may provide predictive outputs data that indicate a trial outcome and a probability of success based on the trial parameters and historical data acquired form the database 103. Note that the AI/ML module 107 may be implemented on the TP server node 102. The TP server node 102 may process the predictive outputs data received from the AI/ML module 107 to generate a trial outcome report.

While this example describes in detail only one TP server node 102, multiple such nodes may be connected to the network. It should be understood that the TP server node 102 may include additional components and that some of the components described herein may be removed and/or modified without departing from a scope of the TP server node 102 disclosed herein. The TP server node 102 may be a computing device or a server computer, or the like, and may include a processor 204, which may be a semiconductor-based microprocessor, a central processing unit (CPU), graphics processing unit (GPU), an application specific integrated circuit (ASIC), a field-programmable gate array (FPGA), and/or another hardware device. Although a single processor 204 is depicted, it should be understood that the TP server node 102 may include multiple processors, multiple cores, or the like, without departing from the scope of the TP server node 102 system.

The TP server node 102 may also include a non-transitory computer readable medium 212 that may have stored thereon machine-readable instructions executable by the processor 205. Examples of the machine-readable instructions are shown as 214-222 and are further discussed below. Examples of the non-transitory computer readable medium 212 may include an electronic, magnetic, optical, or other physical storage device that contains or stores executable instructions. For example, the non-transitory computer readable medium 212 may be a Random-Access memory (RAM), an Electrically Erasable Programmable Read-Only Memory (EEPROM), a hard disk, a solid-state drive, an optical disc, or other type of storage device.

The processor 204 may fetch, decode, and execute the machine-readable instructions 214 to receive a clinical trial (CT) data. The processor 204 may fetch, decode, and execute the machine-readable instructions 216 to parse the CT data to derive drug molecules data, disease information data, and trial protocols data. The processor 204 may fetch, decode, and execute the machine-readable instructions 218 to encode the drug molecules data, the disease information data, and the trial protocols data into corresponding embeddings. The processor 204 may fetch, decode, and execute the machine-readable instructions 220 to generate knowledge pre-trained embeddings using external knowledge data. The processor 204 may fetch, decode, and execute the machine-readable instructions 222 to provide the knowledge pre-trained embeddings to the ML module for prediction of the CT outcome.

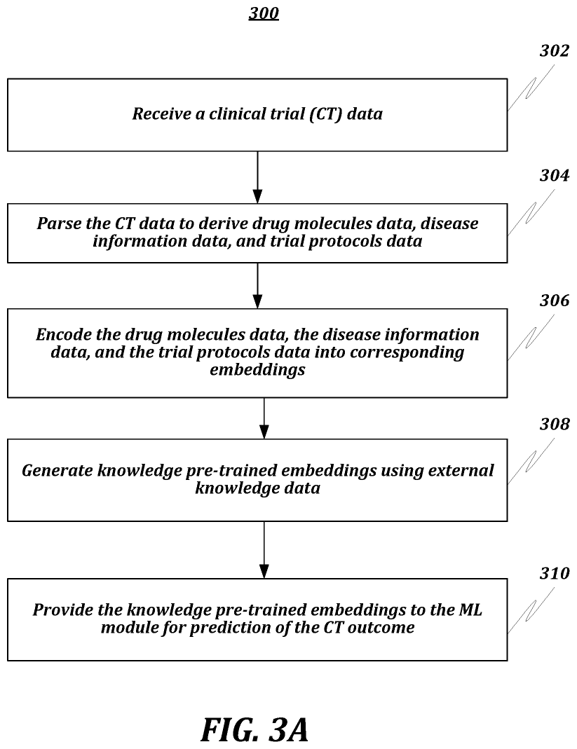

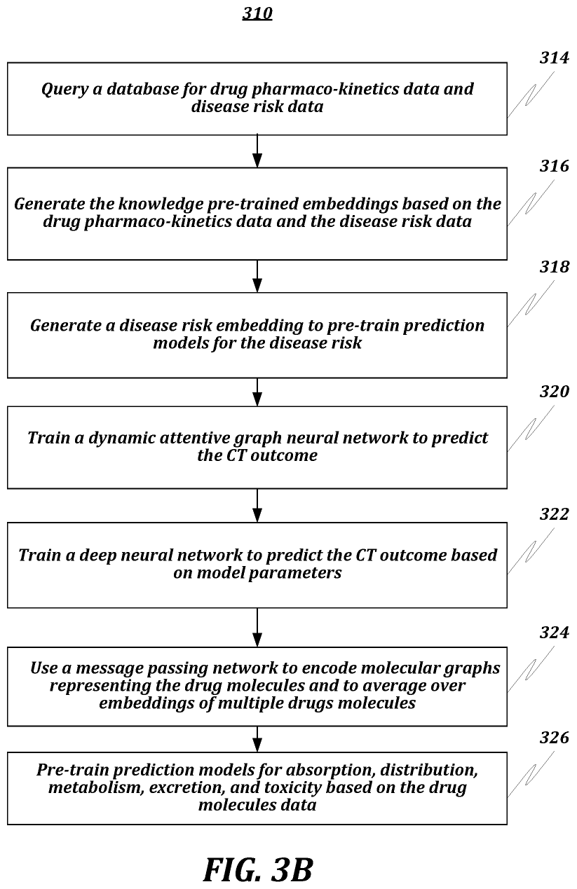

FIG. 3A illustrates a flowchart of a method for an intelligent AI-based prediction of a clinical trial outcome consistent with the present disclosure.

Referring to FIG. 3A, the method 300 may include one or more of the steps described below. FIG. 3A illustrates a flow chart of an example method executed by the TP server 102 (see FIG. 2). It should be understood that method 300 depicted in FIG. 3A may include additional operations and that some of the operations described therein may be removed and/or modified without departing from the scope of the method 300. The description of the method 300 is also made with reference to the features depicted in FIG. 2 for purposes of illustration. Particularly, the processor 204 of the TP server 102 may execute some or all of the operations included in the method 300.

With reference to FIG. 3A, at block 302, the processor 204 may receive a clinical trial (CT) data. At block 304, the processor 204 may parse the CT data to derive drug molecules data, disease information data, and trial protocols data. At block 306, the processor 204 may encode the drug molecules data, the disease information data, and the trial protocols data into corresponding embeddings. At block 308, the processor 204 may generate knowledge pre-trained embeddings using external knowledge data. At block 310, the processor 204 may provide the knowledge pre-trained embeddings to the ML module 107 for prediction of the CT outcome. As discussed above, the ML module 107 may generated the predictive models 108.

FIG. 3B illustrates a further flowchart of a method for an automated AI-based prediction of a clinical trial outcome consistent with the present disclosure. Referring to FIG. 3B, the method 310 may include one or more of the steps described below. FIG. 3B illustrates a flow chart of an example method executed by the TP server 102 (see FIG. 2). It should be understood that method 310 depicted in FIG. 3B may include additional operations and that some of the operations described therein may be removed and/or modified without departing from the scope of the method 310. The description of the method 310 is also made with reference to the features depicted in FIG. 2 for purposes of illustration. Particularly, the processor 204 of the TP server 102 may execute some or all of the operations included in the method 300.

With reference to FIG. 3B, at block 314, the processor 204 may query a database 103 (see FIG. 2) for drug pharmaco-kinetics data and disease risk data. At block 316, the processor 204 may generate the knowledge pre-trained embeddings based on the drug pharmaco-kinetics data and the disease risk data. At block 318, the processor 204 may generate a disease risk embedding to pre-train prediction models for the disease risk. At block 320, the processor 204 may train a dynamic attentive graph neural network to predict the CT outcome. At block 322, the processor 204 may train a deep neural network y=f_{θ}(M,D,C) to predict the CT outcome based on model parameters θ. At block 324, the processor 204 may use a message passing network to encode molecular graphs representing the drug molecules and to average over embeddings of multiple drugs molecules. At block 326, the processor 204 may pre-train prediction models for absorption, distribution, metabolism, excretion, and toxicity based on the drug molecules data.

In one disclosed embodiment, the clinical trial outcome prediction model may be generated by an AI/ML module 107 that may use training data sets to improve accuracy of the prediction of trial success/failure. The parameters used in training data sets may be stored in a centralized database 103 (FIG. 1A). In one embodiment, a neural network may be used for trial participation modeling and prediction.

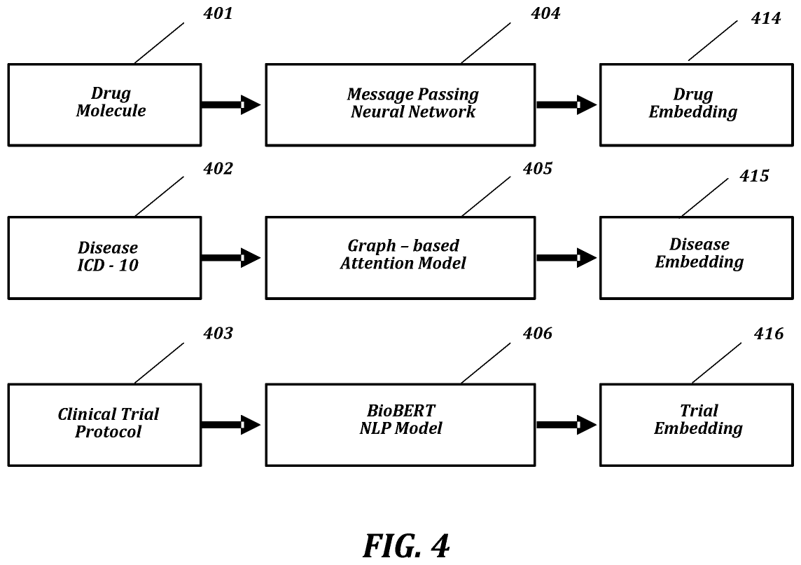

FIG. 4 illustrates a diagram of a clinical trial outcome prediction process consistent with the present disclosure.

According to the disclosed embodiments, problem formulation may be implemented as follows. A clinical trial aims to validate the safety and efficacy of a treatment set towards a target disease set on a patient group.

Definition 1 (Treatment Set). In a trial, the treatment set includes one or multiple drug candidates, denoted by M={m₁, . . . , m_{N}_{ₘ}}. The system of FIG. 1 restricts to trials that aim at discovering new indications of drug candidates. Other trials that involve surgeries or devices are not considered. Drug molecule 401 is used.

Definition 2 (Target Disease Set). Each trial targets one or more diseases 402, denoted D={d₁, . . . , d_{N}_{_{d}}}, dᵢ represents the disease code of the i-th disease. An ICD-10 code is used based on the 10th revision of the International Statistical Classification of Diseases 402.

Definition 3 (Trial Protocol). Each clinical trial protocol 403 consists of inclusion and exclusion criteria in a text format, describing the characteristics of desired patients for the trial, such as age, gender, medical history, target disease conditions, and current health status. It is denoted as C=[c₁, . . . , c_{N}_{ₚ}].

Problem 1 (Clinical Trial Outcome Prediction). The trial outcome is a binary label y, y=1 indicates trial success (i.e., the trial received approval status) and 0 indicates failure (i.e., all the other status). In this trial outcome prediction task, the system trains a deep neural network y=f_{θ}(M,D,C) to predict the outcome based on the model parameters θ.

As discussed above, an input embedding module encodes multi-modal data, including drug molecules, disease information, and trial protocols to embeddings. Next, these embeddings are fed into a knowledge embedding module to generate knowledge embeddings pre-trained using external knowledge including drug pharmaco-kinetics and disease risk data. Then, the interaction graph module connects all the embeddings via domain knowledge to fully capture various trial components and their complex relations as well as their influences on trial outcomes. Based on that, the HINT learns (i.e., trains) dynamic attentive graph neural network to predict trial outcome.

An input embedding module may learn embedding from drug molecules; disease information; trial protocols.

The drug molecules are represented by molecule graphs. The disclosed embodiments may use message passing network (MPN) 404 to encode molecular graphs and average over embeddings of all the molecules in the treatment set as the treatment embedding:

hₘ=mean([MPN(m₁), . . . ,MPN(m_{N}_{ₘ})])∈R^{d},

where M={m₁, . . . ,m_{N}_{ₘ}} is the drug treatment set.

Disease information also affects the outcome. For example, drugs in oncology have much lower approval rates than ones in infectious diseases. The disease information comes from its description and its corresponding ontology, such as disease hierarchies like International Classification of Diseases (ICD). D={d₁, . . . , d_{N}_{_{d}}}(Definition 2), the diseases 402 in the trial are represented as:

h_{d}=mean([GRAM(d₁), . . . ,GRAM(d_{N}_{_{d}})])∈R^{d},

where GRAM(dᵢ) represents embedding of dᵢ using GRAM (graph-based attention model 405), which leverages the hierarchical information inherent to medical ontologies.

The trial protocol describes eligibility criteria, i.e., patient recruitment requirements. Each inclusion or exclusion criteria corresponds to a sentence. Clinical-BERT 406 may be applied to get the sentence representation and average over all the sentences:

hₚ=mean([ClinicalBERT(c₁), . . . ,ClinicalBERT(c_{N}_{ₚ})])∈R^{d}.

One special challenge associated with clinical trial data is that there can be missing data on molecular information due to proprietary information. This poses a problem since representation of many nodes depends on the molecular information. It has been observed that there exists high correlation between the drug molecule, disease and protocol features. Corresponding embeddings 414, 415 and 416 are generated.

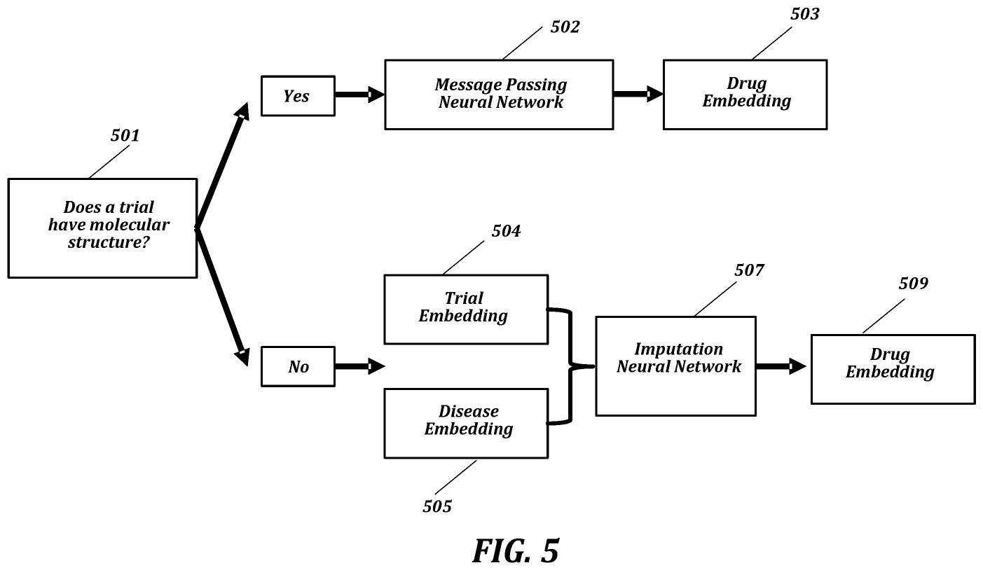

FIG. 5 illustrates a diagram of drug embedding for clinical trial outcome prediction consistent with the present disclosure.

The disclosed embodiments provide a missing data to an imputation neural network module 507 based on learning embeddings that capture inter-modal correlations and intra-modal distribution.

If a trial has a molecular structure at block 501, a message passing neural network 502 is used to generate drug embedding 503. Otherwise, trial embedding 504 and disease embedding 505 are fed to the imputation neural network module 507 to generate the drug embedding 509.

In particular, the imputation module IMP(⋅) uses disease and protocol embedding (h_{d},hₚ) to recover molecular embedding hₘ:

[symbol]

=IMP(h_{d},hₚ)

Here, the system adopts MSE (Mean Square Error) loss as the learning objective to minimize the distance between the ground truth molecule embedding and predicted molecule embedding.

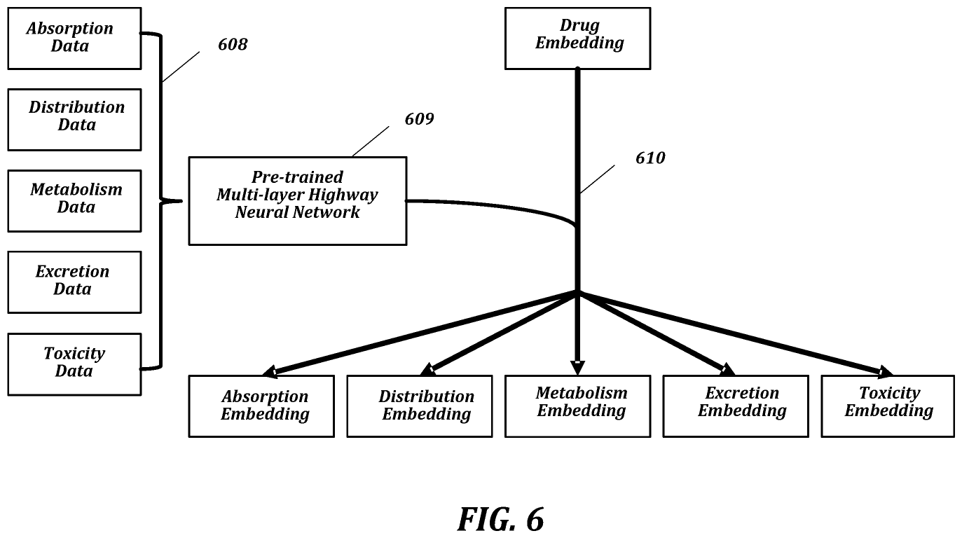

FIG. 6 illustrates a further diagram of drug embedding consistent with the present disclosure.

In case of pharmaco-kinetics knowledge, the embeddings are pre-trained using the pharmaco-kinetics (PK) knowledge about how the body reacts to the intake of a drug. The success of a clinical trial highly depends on factors including drugs' pharmaco-kinetics properties and disease risk. Specifically, the HINT system may leverage various public PK experimental scores. Based on this information, the system may pre-train on prediction models for Absorption, Distribution, Metabolism, Excretion, Toxicity (ADMET) properties 608, which are used to observe how a drug interacts with the body. Absorption describes how drugs travels from administration site to the site of action. Distribution measures the drug's ability to move through the bloodstream. Metabolism measures the duration of a drug's efficacy. Excretion measures how much of drug's toxic components can be removed from the body. Toxicity model measures damage to the body. For each pharmaco-kinetics property 608, binary labels may be used to indicate whether the drug is desirable in this property. Concretely, for each *∈{A,D,M,E,T}, the system pre-trains a knowledge node based on the label y_{*}∈{0,1} as follows:

h_{*}=X_{*}(hₘ)∈R^{d}, y_{*}=Sigmoid(FC(h_{*})), min−y_{*} log ŷ_{*}−(1−y_{*})log(1−y_{*}),

where X is a two-layer highway network 609. FC is a fully-connected layer. Binary cross-entropy is a loss criteria. After training, the drug in each trial can be fed to the pre-trained model to obtain absorption/distribution/metabolism/excretion/toxicity embeddings 610.

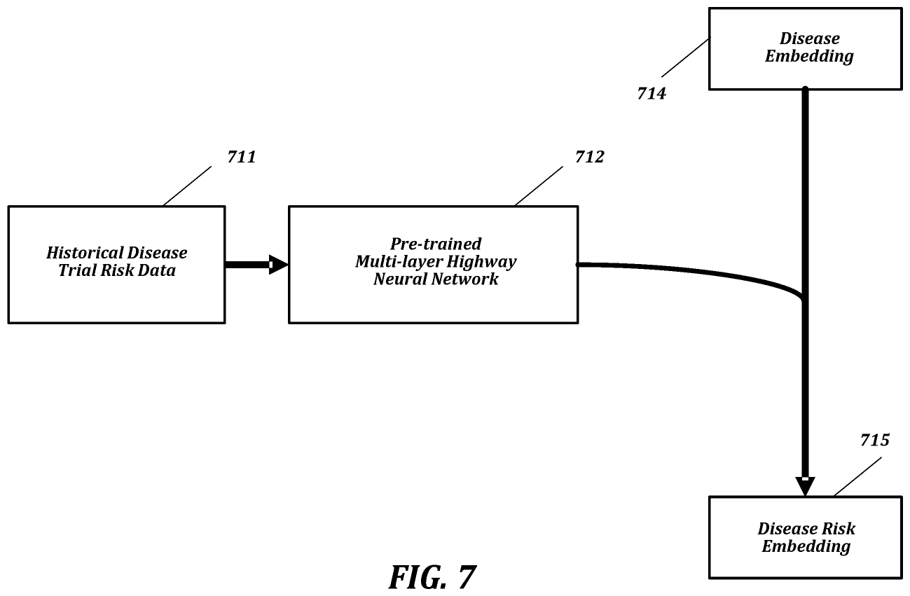

FIG. 7 illustrates a diagram of disease embedding for clinical trial outcome prediction consistent with the present disclosure.

With respect to a disease risk, the disclosed embodiments consider the knowledge distilled from historical trials of the target diseases. The historical trial success rate 411 may be used for the disease. As detailed statistics for trial success rate of each disease at different trial phases are widely available, the system considers that as the supervision signal to train the disease risk prediction model based on multi-layer highway neural network 712:

h_{R}=R(h_{d}), ŷ_{R}=Sigmoid(FC(h_{R})), min−y_{R} log [symbol] −(1−y_{R})log(1− [symbol] ),

where h_{d} is the disease embedding 714 defined above, h_{R} is disease risk embedding, y_{R}∈{0,1} is the risk label. Cross-entropy loss is minimized. After training, the disease information in the trial is fed to the pretrained model 712 to obtain disease risk embedding 715.

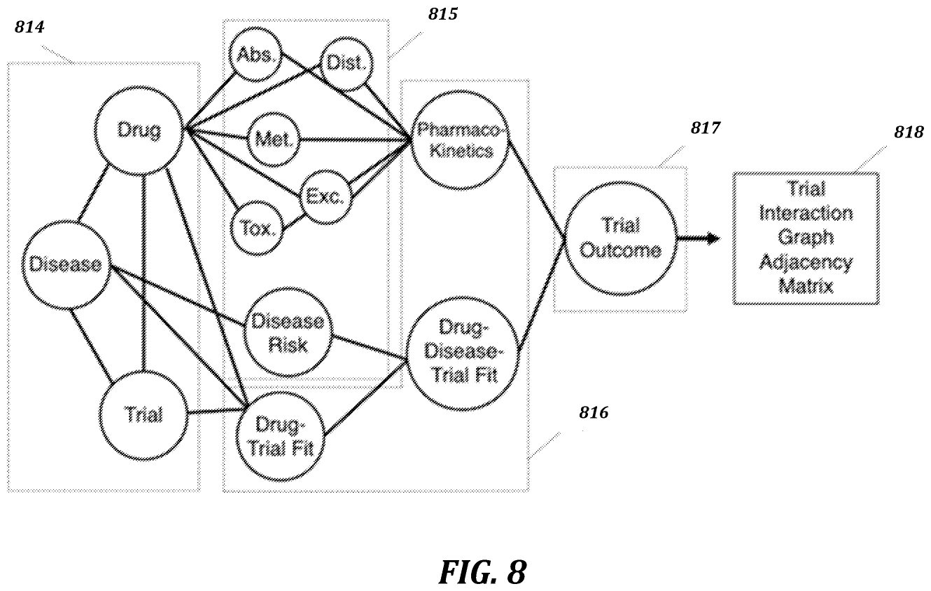

FIG. 8 illustrates a diagram of connection of different nodes used for node embedding for the trial outcome prediction interaction graph consistent with the present disclosure.

In one embodiment, a hierarchical interaction graph G may be constructed to connect input data sources and important factors affecting clinical trial outcomes. The graph 818 reflects the real-world trial development process and consists of four tiers of nodes that are connected between tiers:

Input nodes include drugs, diseases and protocols 814 with node features of input embedding hₘ,h_{d},hₚ∈R^{d};

External knowledge nodes include ADMET embeddings 815 h_{A},h_{D},hₘ,h_{E},h_{T}∈R^{d} and disease risk embedding h_{R}∈R^{d}. These representations are pretrained on external knowledge.

Aggregation nodes include:

(a) Interaction node h_{I} connecting disease h_{d}, drug molecules hₘ and protocols hₚ;

(b) Pharmaco-Kinetics node h_{PK} connecting ADMET embeddings

h_{A},h_{D},h_{M},h_{E},h_{T}∈R^{d};

(c) augmented interaction node h_{V} that augments interaction node h_{I} using disease risk node h_{R}.

Prediction node: h_{pred} connects pharmaco-kinetics node h_{PK} and augmented interaction node h_{V} to make the prediction.

According to the disclosed embodiments, the aggregation nodes are implemented as follows. The PK (Pharmaco-Kinetics) node 816 is to gather information about ADMET properties. PK (Pharmaco-Kinetics) embedding is obtained by:

h_{PK}=f_{K}([h_{A},h_{D},h_{M},h_{E},h_{T}]^{T})∈R^{d},

where f_{K}(⋅) a one-layer fully-connected layer followed by a two-layer highway network.

Then, the system models the interaction among drug molecule, disease and protocol, the interaction node embedding is:

h_{I}=f_{I}([hₘ,h_{d},hₚ]^{T})∈R^{d},

f_{I}(⋅) has the same architecture as f_{K}(⋅).

The Augmented interaction node combines (i) trial risk of the target disease h_{R} and (ii) the interaction node h_{I}:

h_{V}=f_{V}([h_{R},h_{I}]^{T})∈R^{d},

where f_{V} uses the same architecture as f_{K}(⋅), f_{I}(⋅).

The Prediction node summarizes pharmaco-kinetics and augmented interaction:

h_{pred}=fₚ([h_{PK},h_{V}]^{T})∈R^{d},

where fₚ uses the same architecture as f_{I}, f_{V}.

The Dynamic Attentive Graph Neural Network is implemented as follows. The trial embeddings provide initial representations of different trial components and their interactions via a graph. To further enhance interaction, a dynamic attentive graph neural network is designed. Mathematically, in interaction graph G, nodes are trial components, edges are the relations among them.

A∈{0,1}^{K×K} denotes the adjacency matrix 818 based on trial outcome 817. The matrix 818 of G, K=13 is the node number. Node embeddings H⁽⁰⁾ are initialized by stacking representation of all the components:

H⁽⁰⁾=[hₘ,h_{d},hₚ,h_{A},h_{D},h_{M},h_{E},h_{T},h_{R},h_{PK},h_{I},h_{V},h_{pred}]^{T}∈R^{K×d}.

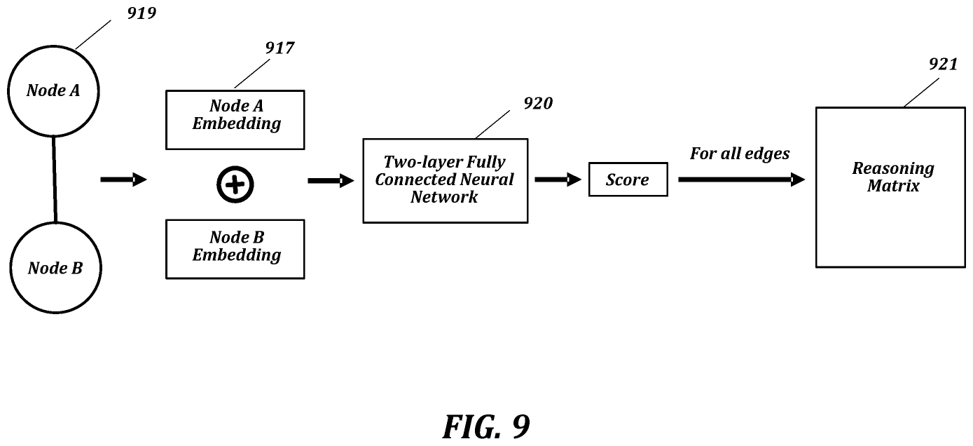

FIG. 9 illustrates a diagram for generation of a reasoning matrix associated with predicted trial outcome consistent with the present disclosure.

As discussed above, different from conventional GCN, the system employs a dynamic layer-independent attentive matrix V∈R₊^{K×K}, V_{i,j} which measures the importance of the edge between i-th and j-th nodes 819. It is parameterized by a two-layer fully-connected neural network 820 with ReLU and Sigmoid activation function in the hidden and output layer:

V_{i,j}=f_{V}([hᵢ,hⱼ]^{T})∈R₊.

The two-layer fully-connected neural network 620 is fed node embeddings 817.

V is the reasoning matrix 821 where each entry corresponds to the reasoning score among each trial component. It is then element-wisely multiplied to adjacency matrix A so that it assigns weights to edges in interaction graph G.

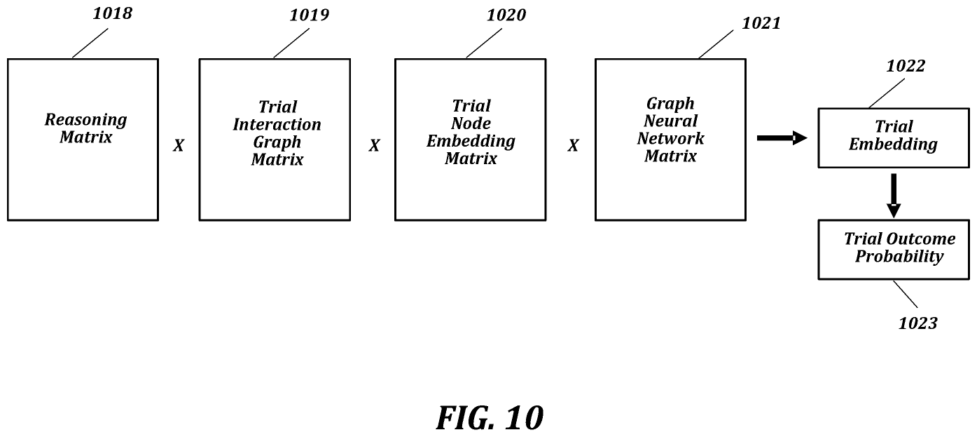

FIG. 10 illustrates a diagram for determining a trial outcome probability based on trial embedding consistent with the present disclosure;

The disclosed embodiment enhances the interaction between nodes using graph convolutional network (GCN) 1022. The updating rule for the l-th layer is:

H^{(l)}=RELU(B^{(l)}+(V⊙A)H^{(l-1)}W^{(l)}, l=1, . . . ,L,

where L is GCN depth. In the l-th layer, H^{(l)}∈R^{K×d} is node embeddings, B^{(l)}, W^{(l)} are the bias/weight parameter, ⊙ represents element-wise multiplication. Different from conventional GCN, the system employs a dynamic layer-independent attentive matrix V∈R₊^{K×K}, V_{i,j} which measures the importance of the edge between i-th and j-th nodes 919 (see FIG. 6).

The prediction and training are implemented as follows. After GNN message-passing, updated representations for trial components are obtained. To generate the trial success (probability) prediction 1023 ŷ, the last-layer (L-th) representation of the prediction node is fed into a one-layer fully-connected network with sigmoid function, and leverage binary cross-entropy loss to guide training.

ŷ=Sigmoid(FC(h_{pred}^{(L)})), min−y log ŷ−(1−y)log(1−ŷ),

where y∈{0,1} is the ground truth. The HINT is trained in an end-to-end manner.

The trial embedding 1022 is based on matrixes 1018-1021.

According to the disclosed embodiments, Data Preparation is implemented as follows. As there is no public trial outcome prediction dataset available, data may be collected from several public data sources. For each trial registered in clinicaltrials.gov, the following is collected:

1) molecule information for tested drugs, available at DrugBank (www.drugbank.com);

2) ICD-10 codes, public API (clinicaltables.nlm.nih.gov) can convert the disease description into ICD-10 code;

3) trial protocol, i.e., eligibility criteria, available in clinicaltrials.gov,

4) trial outcome, a binary indicator of trial success (1) or failure (0), available at clinicaltrials.gov.

Trials that have statistical analysis results in terms of p-value for the primary outcome measure may be selected. The trial is labelled as success if p-value is less than 0.05 and negative if p-value is higher than 0.05. It is validated by an internal dataset.

Additionally, auxiliary the following data is provided:

Pharmaco-kinetics data, which consists of wet lab experiment results for five important PK tasks and available at moleculenet.ai;

Disease risk data, including the past disease trial history success rate, also available at clinicaltrials.gov.

Experimental setting may be implemented as follows. Evaluation settings may consider two realistic setups:

(1) Phase-level evaluation predicts the outcome of single-phase study. Since each phase has different goals (e.g., Phase I is for safety whereas Phase II and III are for efficacy), experiments for Phase I, II and III may be conducted individually. The test datasets are created using the FDA guideline on the success-failure ratio for each phase, specifically 70% success rate for Phase I, 33% for Phase II and 30% for Phase III.

(2) Indication-level evaluation predicts if the drug can pass all three phases for market approval. To imitate it, all phase studies assembled related to the drug and disease of the study and then use the latest phase protocol as the input to our model. Drugs that have Phase III success are labelled positive and other drugs that fail in any of the three phases are labelled negative. Data statistics are shown in Table 1 (see appendix ii).

Data split is implemented as follows. The dataset is split based on a registration date. The earlier trials are used for learning while the later trials are used for inference. For example, for Phase I dataset, the model is trained using the trials before Aug. 13, 2014 and inference is made using the trials after that date, as shown in Table 1 (see appendix ii).

Metrics are implemented as follows: apply (i) PR-AUC (Precision-Recall Area Under Curve); (ii) F1 score; (iii) ROC-AUC (Area Under the Receiver Operating Characteristic Curve) to measure the prediction accuracy. The hypothesis testing results measured by p-value are reported to showcase the statistical significance of our method over the best baseline method.

Baselines—HINT is compared with several baselines, including both conventional machine learning models and deep learning methods, including:

(1) LR (Logistic Regression);

(2) RF (Random Forest);

(3) AdaBoost (Adaptive Boosting);

(4) FFNN (Feed-Forward Neural Network);

(5) DeepEnroll;

(6) COMPOSE (cross-modal pseudo-siamese network).

Experimental Result.

Exp 1. Phase-level prediction focuses on predicting the outcome for a particular phase. In general, phase I tests the toxicity and side effects of the drug; phase II explores the efficacy of the drug (i.e., if the drug works); phase III focuses on drug effectiveness (whether the drug is better than the standard practice/placebo for a well-defined population). For each phase, we train a separate model.

From the results in Table 2 (see appendix ii), it is easy to see deep learning-based approaches including FFNN, DeepEnroll, COMPOSE and HINT outperforms conventional machine learning approaches (LR, RF, XGBoost, AdaBoost) significantly in outcome prediction for all the three phases, thus validating the benefit of deep learning methods for clinical trial outcome prediction. Among all the deep learning methods, HINT performs best with 0.837 F1 for phase I, 0.638 for phase II and 0.683 for phase III. Compared with the strongest baseline (COMPOSE), which is also deep learning approach that uses all the features, HINT achieved 12.2%, 5.6%, 5.6% relatively improvement in terms of PR-AUC and 9.0%, 4.2%, 4.6% relative improvement in terms of F1 score.

The reason is that HINT incorporates insightful multimodal data embedding and finer-grained interaction between multimodal data and trial components (i.e., node in interaction graph). The full HINT performs better than the variant without pre-training model (HINT-Pretrain) and the one without GNN model (HINT-GNN) in both phase I and II scenarios. This observation confirmed the importance of all modules in HINT. When comparing the prediction performance across phase I-Ill, it is found that phase I achieves highest accuracy for almost all the methods while phase II is most challenging with lowest accuracy. This result is consistent with historical trials statistics and reported accuracy of machine learning models on these tasks.

Exp 2. Indication-level prediction focuses on whether the indication of a drug will be approved (pass all 3 phases). A separate model is built using a combined dataset where the successful trials are the ones passed phase III (plus the ones reached phase IV) while the failed trials are the ones that failed in any phase from I to III. The results are presented in Table 3 (see appendix ii). Similar trends have been observed. In particular, HINT performs the best with 0.670 PRAUC, 0.762 F1 and 0.783 ROC-AUC which achieves 6.5%, 5.8%, 7.0% relative improvements on PR-AUC, F1 and ROC-AUC over the strongest baseline (COMPOSE).

The above embodiments of the present disclosure may be implemented in hardware, in a computer-readable instructions executed by a processor, in firmware, or in a combination of the above. The computer computer-readable instructions may be embodied on a computer-readable medium, such as a storage medium. For example, the computer computer-readable instructions may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

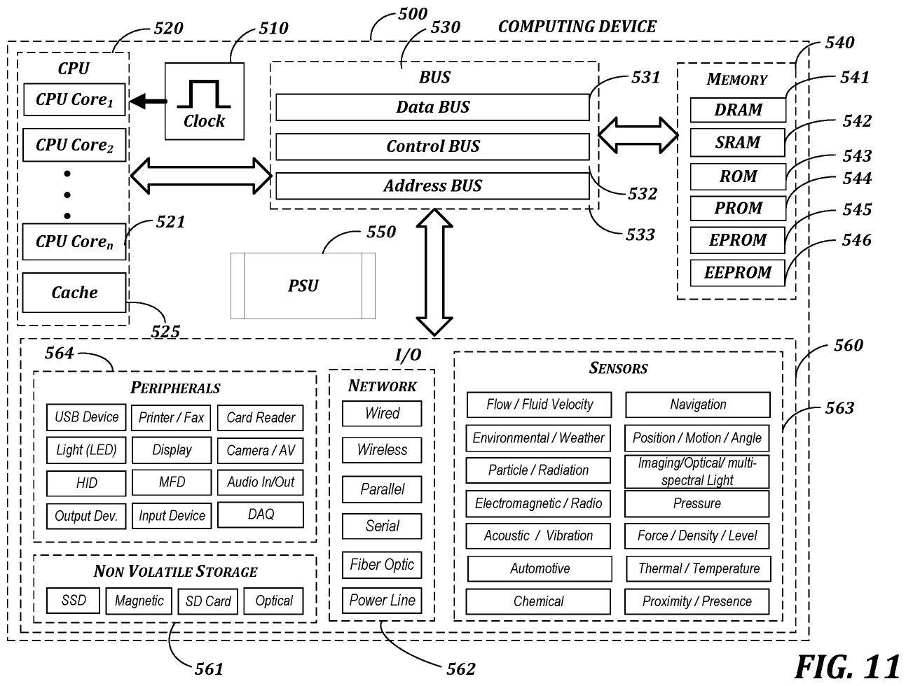

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative embodiment, the processor and the storage medium may reside as discrete components. For example, FIG. 11 illustrates an example computing device (e.g., a server node) 500, which may represent or be integrated in any of the above-described components, etc.

FIG. 11 illustrates a block diagram of a system including computing device 500. The computing device 500 may comprise, but not be limited to the following:

Mobile computing device, such as, but is not limited to, a laptop, a tablet, a smartphone, a drone, a wearable, an embedded device, a handheld device, an Arduino, an industrial device, or a remotely operable recording device;

A supercomputer, an exa-scale supercomputer, a mainframe, or a quantum computer;

A minicomputer, wherein the minicomputer computing device comprises, but is not limited to, an IBM AS500/iSeries/System I, A DEC VAX/PDP, a HP3000, a Honeywell-Bull DPS, a Texas Instruments TI-990, or a Wang Laboratories VS Series;

A microcomputer, wherein the microcomputer computing device comprises, but is not limited to, a server, wherein a server may be rack mounted, a workstation, an industrial device, a raspberry pi, a desktop, or an embedded device;

The trial prediction (TP) server node 102 (see FIG. 2) may be hosted on a centralized server or on a cloud computing service. Although method 300 has been described to be performed by the TP server node 102 implemented on a computing device 500, it should be understood that, in some embodiments, different operations may be performed by a plurality of the computing devices 500 in operative communication at least one network.

Embodiments of the present disclosure may comprise a computing device having a central processing unit (CPU) 520, a bus 530, a memory unit 550, a power supply unit (PSU) 550, and one or more Input/Output (I/O) units. The CPU 520 coupled to the memory unit 550 and the plurality of I/O units 560 via the bus 530, all of which are powered by the PSU 550. It should be understood that, in some embodiments, each disclosed unit may actually be a plurality of such units for the purposes of redundancy, high availability, and/or performance. The combination of the presently disclosed units is configured to perform the stages any method disclosed herein.

Consistent with an embodiment of the disclosure, the aforementioned CPU 520, the bus 530, the memory unit 550, a PSU 550, and the plurality of I/O units 560 may be implemented in a computing device, such as computing device 500. Any suitable combination of hardware, software, or firmware may be used to implement the aforementioned units. For example, the CPU 520, the bus 530, and the memory unit 550 may be implemented with computing device 500 or any of other computing devices 500, in combination with computing device 500. The aforementioned system, device, and components are examples and other systems, devices, and components may comprise the aforementioned CPU 520, the bus 530, the memory unit 550, consistent with embodiments of the disclosure.

At least one computing device 500 may be embodied as any of the computing elements illustrated in all of the attached figures, including the design server node 102 (FIG. 2). A computing device 500 does not need to be electronic, nor even have a CPU 520, nor bus 530, nor memory unit 550. The definition of the computing device 500 to a person having ordinary skill in the art is “A device that computes, especially a programmable [usually] electronic machine that performs high-speed mathematical or logical operations or that assembles, stores, correlates, or otherwise processes information.” Any device which processes information qualifies as a computing device 500, especially if the processing is purposeful.

With reference to FIG. 11, a system consistent with an embodiment of the disclosure may include a computing device, such as computing device 500. In a basic configuration, computing device 500 may include at least one clock module 110, at least one CPU 520, at least one bus 530, and at least one memory unit 550, at least one PSU 550, and at least one I/O 560 module, wherein I/O module may be comprised of, but not limited to a non-volatile storage sub-module 561, a communication sub-module 562, a sensors sub-module 563, and a peripherals sub-module 565.

A system consistent with an embodiment of the disclosure the computing device 500 may include the clock module 510 may be known to a person having ordinary skill in the art as a clock generator, which produces clock signals. Clock signal is a particular type of signal that oscillates between a high and a low state and is used like a metronome to coordinate actions of digital circuits. Most integrated circuits (ICs) of sufficient complexity use a clock signal in order to synchronize different parts of the circuit, cycling at a rate slower than the worst-case internal propagation delays. The preeminent example of the aforementioned integrated circuit is the CPU 520, the central component of modern computers, which relies on a clock. The only exceptions are asynchronous circuits such as asynchronous CPUs. The clock 510 can comprise a plurality of embodiments, such as, but not limited to, single-phase clock which transmits all clock signals on effectively 1 wire, two-phase clock which distributes clock signals on two wires, each with non-overlapping pulses, and four-phase clock which distributes clock signals on 5 wires.

Many computing devices 500 use a “clock multiplier” which multiplies a lower frequency external clock to the appropriate clock rate of the CPU 520. This allows the CPU 520 to operate at a much higher frequency than the rest of the computer, which affords performance gains in situations where the CPU 520 does not need to wait on an external factor (like memory 550 or input/output 560). Some embodiments of the clock 510 may include dynamic frequency change, where, the time between clock edges can vary widely from one edge to the next and back again.

A system consistent with an embodiment of the disclosure the computing device 500 may include the CPU unit 520 comprising at least one CPU Core 521. A plurality of CPU cores 521 may comprise identical CPU cores 521, such as, but not limited to, homogeneous multi-core systems. It is also possible for the plurality of CPU cores 521 to comprise different CPU cores 521, such as, but not limited to, heterogeneous multi-core systems, big.LITTLE systems and some AMD accelerated processing units (APU). The CPU unit 520 reads and executes program instructions which may be used across many application domains, for example, but not limited to, general purpose computing, embedded computing, network computing, digital signal processing (DSP), and graphics processing (GPU). The CPU unit 520 may run multiple instructions on separate CPU cores 521 at the same time. The CPU unit 520 may be integrated into at least one of a single integrated circuit die and multiple dies in a single chip package. The single integrated circuit die and multiple dies in a single chip package may contain a plurality of other aspects of the computing device 500, for example, but not limited to, the clock 510, the CPU 520, the bus 530, the memory 550, and I/O 560.

The CPU unit 520 may contain cache 522 such as, but not limited to, a level 1 cache, level 2 cache, level 3 cache or combination thereof. The aforementioned cache 522 may or may not be shared amongst a plurality of CPU cores 521. The cache 522 sharing comprises at least one of message passing and inter-core communication methods may be used for the at least one CPU Core 521 to communicate with the cache 522. The inter-core communication methods may comprise, but not limited to, bus, ring, two-dimensional mesh, and crossbar. The aforementioned CPU unit 520 may employ symmetric multiprocessing (SMP) design.

The plurality of the aforementioned CPU cores 521 may comprise soft microprocessor cores on a single field programmable gate array (FPGA), such as semiconductor intellectual property cores (IP Core). The plurality of CPU cores 521 architecture may be based on at least one of, but not limited to, Complex instruction set computing (CISC), Zero instruction set computing (ZISC), and Reduced instruction set computing (RISC). At least one of the performance-enhancing methods may be employed by the plurality of the CPU cores 521, for example, but not limited to Instruction-level parallelism (ILP) such as, but not limited to, superscalar pipelining, and Thread-level parallelism (TLP).

Consistent with the embodiments of the present disclosure, the aforementioned computing device 500 may employ a communication system that transfers data between components inside the aforementioned computing device 500, and/or the plurality of computing devices 500. The aforementioned communication system will be known to a person having ordinary skill in the art as a bus 530. The bus 530 may embody internal and/or external plurality of hardware and software components, for example, but not limited to a wire, optical fiber, communication protocols, and any physical arrangement that provides the same logical function as a parallel electrical bus. The bus 530 may comprise at least one of, but not limited to a parallel bus, wherein the parallel bus carry data words in parallel on multiple wires, and a serial bus, wherein the serial bus carry data in bit-serial form. The bus 530 may embody a plurality of topologies, for example, but not limited to, a multidrop/electrical parallel topology, a daisy chain topology, and a connected by switched hubs, such as USB bus. The bus 530 may comprise a plurality of embodiments, for example, but not limited to:

Internal data bus (data bus) 531/Memory bus

Control bus 532

Address bus 533

System Management Bus (SMBus)

Front-Side-Bus (FSB)

External Bus Interface (EBI)Local bus

Expansion busLightning bus

Controller Area Network (CAN bus)

Camera LinkExpressCard

Advanced Technology management Attachment (ATA), including embodiments and derivatives such as, but not limited to, Integrated Drive Electronics (IDE)/Enhanced IDE (EIDE), ATA Packet Interface (ATAPI), Ultra-Direct Memory Access (UDMA), Ultra ATA (UATA)/Parallel ATA (PATA)/Serial ATA (SATA), CompactFlash (CF) interface, Consumer Electronics ATA (CE-ATA)/Fiber Attached Technology Adapted (FATA), Advanced Host Controller Interface (AHCI), SATA Express (SATAe)/External SATA (eSATA), including the powered embodiment eSATAp/Mini-SATA (mSATA), and Next Generation Form Factor (NGFF)/M.2.

Small Computer System Interface (SCSI)/Serial Attached SCSI (SAS)

HyperTransport

InfiniBand

RapidIO

Mobile Industry Processor Interface (MIPI)

Coherent Processor Interface (CAPI)

Plug-n-play

1-Wire

Peripheral Component Interconnect (PCI), including embodiments such as, but not limited to, Accelerated Graphics Port (AGP), Peripheral Component Interconnect eXtended (PCI-X), Peripheral Component Interconnect Express (PCI-e) (e.g., PCI Express Mini Card, PCI Express M.2 [Mini PCIe v2], PCI Express External Cabling [ePCIe], and PCI Express OCuLink [Optical Copper{Cu} Link]), Express Card, AdvancedTCA, AMC, Universal IO, Thunderbolt/Mini DisplayPort, Mobile PCIe (M-PCIe), U.2, and Non-Volatile Memory Express (NVMe)/Non-Volatile Memory Host Controller Interface Specification (NVMHCIS).

Industry Standard Architecture (ISA), including embodiments such as, but not limited to Extended ISA (EISA), PC/XT-bus/PC/AT-bus/PC/105 bus (e.g., PC/105-Plus, PCI/105-Express, PCI/105, and PCI-105), and Low Pin Count (LPC).

Music Instrument Digital Interface (MIDI)

Universal Serial Bus (USB), including embodiments such as, but not limited to, Media Transfer Protocol (MTP)/Mobile High-Definition Link (MHL), Device Firmware Upgrade (DFU), wireless USB, InterChip USB, IEEE 1395 Interface/Firewire, Thunderbolt, and eXtensible Host Controller Interface (xHCI).

Consistent with the embodiments of the present disclosure, the aforementioned computing device 500 may employ hardware integrated circuits that store information for immediate use in the computing device 500, know to the person having ordinary skill in the art as primary storage or memory 550. The memory 550 operates at high speed, distinguishing it from the non-volatile storage sub-module 561, which may be referred to as secondary or tertiary storage, which provides slow-to-access information but offers higher capacities at lower cost. The contents contained in memory 550, may be transferred to secondary storage via techniques such as, but not limited to, virtual memory and swap. The memory 550 may be associated with addressable semiconductor memory, such as integrated circuits consisting of silicon-based transistors, used for example as primary storage but also other purposes in the computing device 500. The memory 550 may comprise a plurality of embodiments, such as, but not limited to volatile memory, non-volatile memory, and semi-volatile memory. It should be understood by a person having ordinary skill in the art that the ensuing are non-limiting examples of the aforementioned memory:

Volatile memory which requires power to maintain stored information, for example, but not limited to, Dynamic Random-Access Memory (DRAM) 551, Static Random-Access Memory (SRAM) 552, CPU Cache memory 525, Advanced Random-Access Memory (A-RAM), and other types of primary storage such as Random-Access Memory (RAM).

Non-volatile memory which can retain stored information even after power is removed, for example, but not limited to, Read-Only Memory (ROM) 553, Programmable ROM (PROM) 555, Erasable PROM (EPROM) 555, Electrically Erasable PROM (EEPROM) 556 (e.g., flash memory and Electrically Alterable PROM [EAPROM]), Mask ROM (MROM), One Time Programable (OTP) ROM/Write Once Read Many (WORM), Ferroelectric RAM (FeRAM), Parallel Random-Access Machine (PRAM), Split-Transfer Torque RAM (STT-RAM), Silicon Oxime Nitride Oxide Silicon (SONOS), Resistive RAM (RRAM), Nano RAM (NRAM), 3D XPoint, Domain-Wall Memory (DWM), and millipede memory.

Semi-volatile memory which may have some limited non-volatile duration after power is removed but loses data after said duration has passed. Semi-volatile memory provides high performance, durability, and other valuable characteristics typically associated with volatile memory, while providing some benefits of true non-volatile memory. The semi-volatile memory may comprise volatile and non-volatile memory and/or volatile memory with battery to provide power after power is removed. The semi-volatile memory may comprise, but not limited to spin-transfer torque RAM (STT-RAM).

Consistent with the embodiments of the present disclosure, the aforementioned computing device 500 may employ the communication system between an information processing system, such as the computing device 500, and the outside world, for example, but not limited to, human, environment, and another computing device 500. The aforementioned communication system will be known to a person having ordinary skill in the art as I/O 560. The I/O module 560 regulates a plurality of inputs and outputs with regard to the computing device 500, wherein the inputs are a plurality of signals and data received by the computing device 500, and the outputs are the plurality of signals and data sent from the computing device 500. The I/O module 560 interfaces a plurality of hardware, such as, but not limited to, non-volatile storage 561, communication devices 562, sensors 563, and peripherals 565. The plurality of hardware is used by the at least one of, but not limited to, human, environment, and another computing device 500 to communicate with the present computing device 500. The I/O module 560 may comprise a plurality of forms, for example, but not limited to channel I/O, port mapped I/O, asynchronous I/O, and Direct Memory Access (DMA).

Consistent with the embodiments of the present disclosure, the aforementioned computing device 500 may employ the non-volatile storage sub-module 561, which may be referred to by a person having ordinary skill in the art as one of secondary storage, external memory, tertiary storage, off-line storage, and auxiliary storage. The non-volatile storage sub-module 561 may not be accessed directly by the CPU 520 without using intermediate area in the memory 550. The non-volatile storage sub-module 561 does not lose data when power is removed and may be two orders of magnitude less costly than storage used in memory module, at the expense of speed and latency. The non-volatile storage sub-module 561 may comprise a plurality of forms, such as, but not limited to, Direct Attached Storage (DAS), Network Attached Storage (NAS), Storage Area Network (SAN), nearline storage, Massive Array of Idle Disks (MAID), Redundant Array of Independent Disks (RAID), device mirroring, off-line storage, and robotic storage. The non-volatile storage sub-module (561) may comprise a plurality of embodiments, such as, but not limited to:

Optical storage, for example, but not limited to, Compact Disk (CD) (CD-ROM/CD-R/CD-RW), Digital Versatile Disk (DVD) (DVD-ROM/DVD-R/DVD+R/DVD-RW/DVD+RW/DVD±RW/DVD+R DL/DVD-RAM/HD-DVD), Blu-ray Disk (BD) (BD-ROM/BD-R/BD-RE/BD-R DL/BD-RE DL), and Ultra-Density Optical (UDO).

Semiconductor storage, for example, but not limited to, flash memory, such as, but not limited to, USB flash drive, Memory card, Subscriber Identity Module (SIM) card, Secure Digital (SD) card, Smart Card, CompactFlash (CF) card, Solid-State Drive (SSD) and memristor.

Magnetic storage such as, but not limited to, Hard Disk Drive (HDD), tape drive, carousel memory, and Card Random-Access Memory (CRAM).

Phase-change memory

Holographic data storage such as Holographic Versatile Disk (HVD).

Molecular Memory

Deoxyribonucleic Acid (DNA) digital data storage

Consistent with the embodiments of the present disclosure, the aforementioned computing device 500 may employ the communication sub-module 562 as a subset of the I/O 560, which may be referred to by a person having ordinary skill in the art as at least one of, but not limited to, computer network, data network, and network. The network allows computing devices 500 to exchange data using connections, which may be known to a person having ordinary skill in the art as data links, between network nodes. The nodes comprise network computer devices 500 that originate, route, and terminate data. The nodes are identified by network addresses and can include a plurality of hosts consistent with the embodiments of a computing device 500. The aforementioned embodiments include, but not limited to personal computers, phones, servers, drones, and networking devices such as, but not limited to, hubs, switches, routers, modems, and firewalls.

Two nodes can be said are networked together, when one computing device 500 is able to exchange information with the other computing device 500, whether or not they have a direct connection with each other. The communication sub-module 562 supports a plurality of applications and services, such as, but not limited to World Wide Web (WWW), digital video and audio, shared use of application and storage computing devices 500, printers/scanners/fax machines, email/online chat/instant messaging, remote control, distributed computing, etc. The network may comprise a plurality of transmission mediums, such as, but not limited to conductive wire, fiber optics, and wireless. The network may comprise a plurality of communications protocols to organize network traffic, wherein application-specific communications protocols are layered, may be known to a person having ordinary skill in the art as carried as payload, over other more general communications protocols. The plurality of communications protocols may comprise, but not limited to, IEEE 802, ethernet, Wireless LAN (WLAN/Wi-Fi), Internet Protocol (IP) suite (e.g., TCP/IP, UDP, Internet Protocol version 5 [IPv5], and Internet Protocol version 6 [IPv6]), Synchronous Optical Networking (SONET)/Synchronous Digital Hierarchy (SDH), Asynchronous Transfer Mode (ATM), and cellular standards (e.g., Global System for Mobile Communications [GSM], General Packet Radio Service [GPRS], Code-Division Multiple Access [CDMA], and Integrated Digital Enhanced Network [IDEN]).

The communication sub-module 562 may comprise a plurality of size, topology, traffic control mechanism and organizational intent. The communication sub-module 562 may comprise a plurality of embodiments, such as, but not limited to:

Wired communications, such as, but not limited to, coaxial cable, phone lines, twisted pair cables (ethernet), and InfiniBand.

Wireless communications, such as, but not limited to, communications satellites, cellular systems, radio frequency/spread spectrum technologies, IEEE 802.11 Wi-Fi, Bluetooth, NFC, free-space optical communications, terrestrial microwave, and Infrared (IR) communications. Wherein cellular systems embody technologies such as, but not limited to, 3G, 5G (such as WiMax and LTE), and 5G (short and long wavelength).

Parallel communications, such as, but not limited to, LPT ports.

Serial communications, such as, but not limited to, RS-232 and USB.

Fiber Optic communications, such as, but not limited to, Single-mode optical fiber (SMF) and Multi-mode optical fiber (MMF).

Power Line and wireless communications

The aforementioned network may comprise a plurality of layouts, such as, but not limited to, bus network such as ethernet, star network such as Wi-Fi, ring network, mesh network, fully connected network, and tree network. The network can be characterized by its physical capacity or its organizational purpose. Use of the network, including user authorization and access rights, differ accordingly. The characterization may include, but not limited to nanoscale network, Personal Area Network (PAN), Local Area Network (LAN), Home Area Network (HAN), Storage Area Network (SAN), Campus Area Network (CAN), backbone network, Metropolitan Area Network (MAN), Wide Area Network (WAN), enterprise private network, Virtual Private Network (VPN), and Global Area Network (GAN).

Consistent with the embodiments of the present disclosure, the aforementioned computing device 500 may employ the sensors sub-module 563 as a subset of the I/O 560. The sensors sub-module 563 comprises at least one of the devices, modules, and subsystems whose purpose is to detect events or changes in its environment and send the information to the computing device 500. Sensors are sensitive to the measured property, are not sensitive to any property not measured, but may be encountered in its application, and do not significantly influence the measured property. The sensors sub-module 563 may comprise a plurality of digital devices and analog devices, wherein if an analog device is used, an Analog to Digital (A-to-D) converter must be employed to interface the said device with the computing device 500. The sensors may be subject to a plurality of deviations that limit sensor accuracy. The sensors sub-module 563 may comprise a plurality of embodiments, such as, but not limited to, chemical sensors, automotive sensors, acoustic/sound/vibration sensors, electric current/electric potential/magnetic/radio sensors, environmental/weather/moisture/humidity sensors, flow/fluid velocity sensors, ionizing radiation/particle sensors, navigation sensors, position/angle/displacement/distance/speed/acceleration sensors, imaging/optical/light sensors, pressure sensors, force/density/level sensors, thermal/temperature sensors, and proximity/presence sensors. It should be understood by a person having ordinary skill in the art that the ensuing are non-limiting examples of the aforementioned sensors:

Chemical sensors, such as, but not limited to, breathalyzer, carbon dioxide sensor, carbon monoxide/smoke detector, catalytic bead sensor, chemical field-effect transistor, chemiresistor, electrochemical gas sensor, electronic nose, electrolyte-insulator-semiconductor sensor, energy-dispersive X-ray spectroscopy, fluorescent chloride sensors, holographic sensor, hydrocarbon dew point analyzer, hydrogen sensor, hydrogen sulfide sensor, infrared point sensor, ion-selective electrode, nondispersive infrared sensor, microwave chemistry sensor, nitrogen oxide sensor, olfactometer, optode, oxygen sensor, ozone monitor, pellistor, pH glass electrode, potentiometric sensor, redox electrode, zinc oxide nanorod sensor, and biosensors (such as nano-sensors).

Automotive sensors, such as, but not limited to, air flow meter/mass airflow sensor, air-fuel ratio meter, AFR sensor, blind spot monitor, engine coolant/exhaust gas/cylinder head/transmission fluid temperature sensor, hall effect sensor, wheel/automatic transmission/turbine/vehicle speed sensor, airbag sensors, brake fluid/engine crankcase/fuel/oil/tire pressure sensor, camshaft/crankshaft/throttle position sensor, fuel/oil level sensor, knock sensor, light sensor, MAP sensor, oxygen sensor (o2), parking sensor, radar sensor, torque sensor, variable reluctance sensor, and water-in-fuel sensor.

Acoustic, sound and vibration sensors, such as, but not limited to, microphone, lace sensor (guitar pickup), seismometer, sound locator, geophone, and hydrophone.

Electric current, electric potential, magnetic, and radio sensors, such as, but not limited to, current sensor, Daly detector, electroscope, electron multiplier, faraday cup, galvanometer, hall effect sensor, hall probe, magnetic anomaly detector, magnetometer, magnetoresistance, MEMS magnetic field sensor, metal detector, planar hall sensor, radio direction finder, and voltage detector.

Environmental, weather, moisture, and humidity sensors, such as, but not limited to, actinometer, air pollution sensor, bedwetting alarm, ceilometer, dew warning, electrochemical gas sensor, fish counter, frequency domain sensor, gas detector, hook gauge evaporimeter, humistor, hygrometer, leaf sensor, lysimeter, pyranometer, pyrgeometer, psychrometer, rain gauge, rain sensor, seismometers, SNOTEL, snow gauge, soil moisture sensor, stream gauge, and tide gauge.

Flow and fluid velocity sensors, such as, but not limited to, air flow meter, anemometer, flow sensor, gas meter, mass flow sensor, and water meter.

Ionizing radiation and particle sensors, such as, but not limited to, cloud chamber, Geiger counter, Geiger-Muller tube, ionization chamber, neutron detection, proportional counter, scintillation counter, semiconductor detector, and thermoluminescent dosimeter.

Navigation sensors, such as, but not limited to, air speed indicator, altimeter, attitude indicator, depth gauge, fluxgate compass, gyroscope, inertial navigation system, inertial reference unit, magnetic compass, MHD sensor, ring laser gyroscope, turn coordinator, variometer, vibrating structure gyroscope, and yaw rate sensor.

Position, angle, displacement, distance, speed, and acceleration sensors, such as, but not limited to, accelerometer, displacement sensor, flex sensor, free fall sensor, gravimeter, impact sensor, laser rangefinder, LIDAR, odometer, photoelectric sensor, position sensor such as, but not limited to, GPS or Glonass, angular rate sensor, shock detector, ultrasonic sensor, tilt sensor, tachometer, ultra-wideband radar, variable reluctance sensor, and velocity receiver.

Imaging, optical and light sensors, such as, but not limited to, CMOS sensor, LiDAR, multi-spectral light sensor, colorimeter, contact image sensor, electro-optical sensor, infra-red sensor, kinetic inductance detector, LED as light sensor, light-addressable potentiometric sensor, Nichols radiometer, fiber-optic sensors, optical position sensor, thermopile laser sensor, photodetector, photodiode, photomultiplier tubes, phototransistor, photoelectric sensor, photoionization detector, photomultiplier, photoresistor, photoswitch, phototube, scintillometer, Shack-Hartmann, single-photon avalanche diode, superconducting nanowire single-photon detector, transition edge sensor, visible light photon counter, and wavefront sensor.

Pressure sensors, such as, but not limited to, barograph, barometer, boost gauge, bourdon gauge, hot filament ionization gauge, ionization gauge, McLeod gauge, Oscillating U-tube, permanent downhole gauge, piezometer, Pirani gauge, pressure sensor, pressure gauge, tactile sensor, and time pressure gauge.

Force, Density, and Level sensors, such as, but not limited to, bhangmeter, hydrometer, force gauge or force sensor, level sensor, load cell, magnetic level or nuclear density sensor or strain gauge, piezocapacitive pressure sensor, piezoelectric sensor, torque sensor, and viscometer.

Thermal and temperature sensors, such as, but not limited to, bolometer, bimetallic strip, calorimeter, exhaust gas temperature gauge, flame detection/pyrometer, Gardon gauge, Golay cell, heat flux sensor, microbolometer, microwave radiometer, net radiometer, infrared/quartz/resistance thermometer, silicon bandgap temperature sensor, thermistor, and thermocouple.

Proximity and presence sensors, such as, but not limited to, alarm sensor, doppler radar, motion detector, occupancy sensor, proximity sensor, passive infrared sensor, reed switch, stud finder, triangulation sensor, touch switch, and wired glove.

Consistent with the embodiments of the present disclosure, the aforementioned computing device 500 may employ the peripherals sub-module 562 as a subset of the I/O 560. The peripheral sub-module 565 comprises ancillary devices uses to put information into and get information out of the computing device 500. There are 3 categories of devices comprising the peripheral sub-module 565, which exist based on their relationship with the computing device 500, input devices, output devices, and input/output devices. Input devices send at least one of data and instructions to the computing device 500. Input devices can be categorized based on, but not limited to:

Modality of input, such as, but not limited to, mechanical motion, audio, visual, and tactile.

Whether the input is discrete, such as but not limited to, pressing a key, or continuous such as, but not limited to position of a mouse.

The number of degrees of freedom involved, such as, but not limited to, two-dimensional mice vs three-dimensional mice used for Computer-Aided Design (CAD) applications.

Output devices provide output from the computing device 500. Output devices convert electronically generated information into a form that can be presented to humans. Input/output devices perform that perform both input and output functions. It should be understood by a person having ordinary skill in the art that the ensuing are non-limiting embodiments of the aforementioned peripheral sub-module 565:

Input Devices

Human Interface Devices (HID), such as, but not limited to, pointing device (e.g., mouse, touchpad, joystick, touchscreen, game controller/gamepad, remote, light pen, light gun, Wii remote, jog dial, shuttle, and knob), keyboard, graphics tablet, digital pen, gesture recognition devices, magnetic ink character recognition, Sip-and-Puff (SNP) device, and Language Acquisition Device (LAD).

High degree of freedom devices, that require up to six degrees of freedom such as, but not limited to, camera gimbals, Cave Automatic Virtual Environment (CAVE), and virtual reality systems.

Video Input devices are used to digitize images or video from the outside world into the computing device 500. The information can be stored in a multitude of formats depending on the user's requirement. Examples of types of video input devices include, but not limited to, digital camera, digital camcorder, portable media player, web cam, Microsoft Kinect, image scanner, fingerprint scanner, barcode reader, 3D scanner, laser rangefinder, eye gaze tracker, computed tomography, magnetic resonance imaging, positron emission tomography, medical ultrasonography, TV tuner, and iris scanner.

Audio input devices are used to capture sound. In some cases, an audio output device can be used as an input device, in order to capture produced sound. Audio input devices allow a user to send audio signals to the computing device 500 for at least one of processing, recording, and carrying out commands. Devices such as microphones allow users to speak to the computer in order to record a voice message or navigate software. Aside from recording, audio input devices are also used with speech recognition software. Examples of types of audio input devices include, but not limited to microphone, Musical Instrumental Digital Interface (MIDI) devices such as, but not limited to a keyboard, and headset.

Data Acquisition (DAQ) devices convert at least one of analog signals and physical parameters to digital values for processing by the computing device 500. Examples of DAQ devices may include, but not limited to, Analog to Digital Converter (ADC), data logger, signal conditioning circuitry, multiplexer, and Time to Digital Converter (TDC).

Output Devices may further comprise, but not be limited to:

Display devices, which convert electrical information into visual form, such as, but not limited to, monitor, TV, projector, and Computer Output Microfilm (COM). Display devices can use a plurality of underlying technologies, such as, but not limited to, Cathode-Ray Tube (CRT), Thin-Film Transistor (TFT), Liquid Crystal Display (LCD), Organic Light-Emitting Diode (OLED), MicroLED, E Ink Display (ePaper) and Refreshable Braille Display (Braille Terminal).

Printers, such as, but not limited to, inkjet printers, laser printers, 3D printers, solid ink printers and plotters.

Audio and Video (AV) devices, such as, but not limited to, speakers, headphones, amplifiers and lights, which include lamps, strobes, DJ lighting, stage lighting, architectural lighting, special effect lighting, and lasers.

Other devices such as Digital to Analog Converter (DAC)

Input/Output Devices may further comprise, but not be limited to, touchscreens, networking device (e.g., devices disclosed in network 562 sub-module), data storage device (non-volatile storage 561), facsimile (FAX), and graphics/sound cards.

All rights including copyrights in the code included herein are vested in and the property of the Applicant. The Applicant retains and reserves all rights in the code included herein, and grants permission to reproduce the material only in connection with reproduction of the granted patent and for no other purpose.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

### Appendix ii

TABLE 1
Data statistics. During training, we randomly select 15% training samples
for validation. The ratio of learning/inference samples is controlled
to be about 4:1. The trials before the split date are used for learning
while the later trials are used for inference. “Success”, “Fail”
are abbreviations for success and failure, respectively.

| | Train | | Test | | Split |
|---|---|---|---|---|---|
| Setting | success | failure | success | fail | Date |
| Phase I | 702 | 386 | 199 | 113 | Aug. 13, 2014 |
| Phase II | 956 | 1655 | 302 | 487 | Mar. 20, 2014 |
| Phase III | 1,820 | 2,493 | 457 | 684 | Apr. 7, 2014 |
| Indication | 1,864 | 2,922 | 473 | 674 | May 21, 2014 |

TABLE 2
Phase-Level prediction. The hypothesis is that the performance
of COMPOSE (the best baseline) is the same as that of HINT. Student's
T-test is used with significance level alpha as 1% to calculate
the p-values. Empirical studies show that for both PRAUC and F1,
all the p-values are below 0.05 threshold, the null hypothesis
is rejected and the alternative hypothesis are accepted, i.e.,
true means are totally different. These results confirm that HINT
is significantly better than COMPOSE (the best baseline).

| | PR-AUC | F1 | ROC-AUC |
|---|---|---|---|
| Phase I | | | |
| LR | 0.575 ± 0.011 | 0.640 ± 0.013 | 0.630 ± 0.017 |
| RF | 0.640 ± 0.012 | 0.656 ± 0.017 | 0.674 ± 0.013 |
| XGBoost | 0.653 ± 0.015 | 0.671 ± 0.016 | 0.698 ± 0.012 |
| AdaBoost | 0.589 ± 0.010 | 0.612 ± 0.015 | 0.623 ± 0.012 |
| FFNN | 0.643 ± 0.020 | 0.745 ± 0.024 | 0.747 ± 0.026c |
| DeepEnroll | 0.654 ± 0.020 | 0.754 ± 0.019 | 0.750 ± 0.021 |
| COMPOSE | 0.681 ± 0.017 | 0.768 ± 0.019 | 0.766 ± 0.016 |
| HINT-pretrain | 0.701 ± 0.022 | 0.792 ± 0.018 | 0.784 ± 0.020 |
| HINT-GNN | 0.753 ± 0.020 | 0.819 ± 0.014 | 0.813 ± 0.016 |
| HINT | 0.764 ± 0.018 | 0.837 ± 0.012 | 0.817 ± 0.015 |
| p-value | 0.00003 | 0.0001 | 0.0004 |
| Phase II | | | |
| LR | 0.489 ± 0.012 | 0.528 ± 0.018 | 0.600 ± 0.016 |
| RF | 0.578 ± 0.020 | 0.611 ± 0.019 | 0.683 ± 0.022 |
| XGBoost | 0.571 ± 0.020 | 0.608 ± 0.012 | 0.694 ± 0.013 |
| AdaBoost | 0.457 ± 0.014 | 0.515 ± 0.017 | 0.556 ± 0.013 |
| FFNN | 0.555 ± 0.020 | 0.569 ± 0.028 | 0.661 ± 0.021 |
| DeepEnroll | 0.560 ± 0.018 | 0.598 ± 0.020 | 0.742 ± 0.017 |
| COMPOSE | 0.570 ± 0.017 | 0.612 ± 0.015 | 0.743 ± 0.016 |
| HINT-pretrain | 0.583 ± 0.017 | 0.621 ± 0.017 | 0.757 ± 0.018 |
| HINT-GNN | 0.595 ± 0.018 | 0.626 ± 0.017 | 0.751 ± 0.018 |
| HINT | 0.602 ± 0.016 | 0.638 ± 0.015 | 0.761 ± 0.018 |
| p-value | 0.008 | 0.012 | 0.048 |
| Phase III | | | |
| LR | 0.533 ± 0.005 | 0.590 ± 0.009 | 0.642 ± 0.007 |
| RF | 0.554 ± 0.008 | 0.626 ± 0.010 | 0.680 ± 0.011 |
| XGBoost | 0.588 ± 0.013 | 0.621 ± 0.014 | 0.719 ± 0.015 |
| AdaBoost | 0.560 ± 0.012 | 0.590 ± 0.015 | 0.678 ± 0.014 |
| FFNN | 0.576 ± 0.018 | 0.628 ± 0.020 | 0.684 ± 0.018 |
| DeepEnroll | 0.581 ± 0.016 | 0.646 ± 0.020 | 0.699 ± 0.016 |
| COMPOSE | 0.589 ± 0.012 | 0.653 ± 0.016 | 0.715 ± 0.016 |
| HINT-pretrain | 0.599 ± 0.018 | 0.658 ± 0.014 | 0.721 ± 0.017 |
| HINT-GNN | 0.622 ± 0.014 | 0.693 ± 0.018 | 0.759 ± 0.018 |
| HINT | 0.618 ± 0.014 | 0.683 ± 0.016 | 0.726 ± 0.015 |
| p-value | 0.002 | 0.009 | 0.14 |

TABLE 3
indication-level prediction.

| Indication | PR-AUC | F1 | ROC-AUC |
|---|---|---|---|
| LR | 0.579 ± 0.007 | 0.613 ± 0.010 | 0.645 ± 0.009 |
| RF | 0.594 ± 0.012 | 0.627 ± 0.012 | 0.621 ± 0.011 |
| XGBoost | 0.603 ± 0.010 | 0.614 ± 0.012 | 0.645 ± 0.011 |
| AdaBoost | 0.565 ± 0.007 | 0.597 ± 0.011 | 0.632 ± 0.011 |
| FFNN | 0.602 ± 0.017 | 0.673 ± 0.019 | 0.708 ± 0.014 |
| DeepEnroll | 0.616 ± 0.016 | 0.675 ± 0.015 | 0.712 ± 0.013 |
| COMPOSE | 0.629 ± 0.017 | 0.720 ± 0.015 | 0.732 ± 0.014 |
| HINT-pretrain | 0.642 ± 0.015 | 0.731 ± 0.016 | 0.749 ± 0.018 |
| HINT-GNN | 0.653 ± 0.014 | 0.756 ± 0.013 | 0.774 ± 0.014 |
| HINT | 0.670 ± 0.014 | 0.762 ± 0.012 | 0.783 ± 0.017 |
| p-value | 0.002 | 0.001 | 0.0004 |

The following is claimed:

1. A system, comprising:
a processor of a trial prediction (TP) node connected to at least one cloud server node over a network configured to host a machine learning (ML) module;
a memory on which are stored machine-readable instructions that when executed by the processor, cause the processor to:
receive a clinical trial (CT) data,
parse the CT data to derive drug molecules data, disease information data, and trial protocols data,
encode the drug molecules data, the disease information data, and the trial protocols data into corresponding embeddings,
generate knowledge pre-trained embeddings using external knowledge data, and
provide the knowledge pre-trained embeddings to the ML module for prediction of the CT outcome.

2. The system of claim 1, wherein the instructions further cause the processor to query a database for drug pharmaco-kinetics data and disease risk data.

3. The system of claim 2, wherein the instructions further cause the processor to generate the knowledge pre-trained embeddings based on the drug pharmaco-kinetics data and the disease risk data.

4. The system of claim 3, wherein the instructions further cause the processor to generate a disease risk embedding to pre-train prediction models for the disease risk.

5. The system of claim 1, wherein the instructions further cause the processor to train a dynamic attentive graph neural network to predict the CT outcome.

6. The system of claim 1, wherein the instructions further cause the processor to train a deep neural network y=f_{θ}(M,D,C) to predict the CT outcome based on model parameters θ.

7. The system of claim 1, wherein the instructions further cause the processor to use a message passing network to encode molecular graphs representing the drug molecules and to average over embeddings of multiple drugs molecules.

8. The system of claim 1, wherein the instructions further cause the processor to pre-train prediction models for absorption, distribution, metabolism, excretion, and toxicity based on the drug molecules data.

9. A method, comprising:
receiving, by a trial prediction (TP) node, a clinical trial (CT) data;
parsing, by the trial prediction (TP) node, the CT data to derive drug molecules data, disease information data, and trial protocols data;
encoding, by the trial prediction (TP) node, the drug molecules data, the disease information data, and the trial protocols data into corresponding embeddings;
generating, by the trial prediction (TP) node, knowledge pre-trained embeddings using external knowledge data; and
providing, by the trial prediction (TP) node, the knowledge pre-trained embeddings to an ML module for prediction of the CT outcome.

10. The method of claim 9, further comprising querying a database for drug pharmaco-kinetics data and disease risk data.

11. The method of claim 10, further comprising generating the knowledge pre-trained embeddings based on the drug pharmaco-kinetics data and the disease risk data.

12. The method of claim 11, further comprising generating a disease risk embedding to pre-train prediction models for the disease risk.

13. The method of claim 9, further comprising training a dynamic attentive graph neural network to predict the CT outcome.

14. The method of claim 9, further comprising training a deep neural network y=f_{θ}(M,D,C) to predict the CT outcome based on model parameters θ.

15. The method of claim 9, further comprising using a message passing network to encode molecular graphs representing the drug molecules and to average over embeddings of multiple drugs molecules.

16. The method of claim 9, further comprising pre-training prediction models for absorption, distribution, metabolism, excretion, and toxicity based on the drug molecules data.

17. A non-transitory computer readable medium comprising instructions, that when read by a processor, cause the processor to perform:
receiving a clinical trial (CT) data;
parsing the CT data to derive drug molecules data, disease information data, and trial protocols data;
encoding the drug molecules data, the disease information data, and the trial protocols data into corresponding embeddings;
generating knowledge pre-trained embeddings using external knowledge data; and
providing the knowledge pre-trained embeddings to an ML module for prediction of the CT outcome.

18. The non-transitory computer readable medium of claim 17, further comprising instructions, that when read by the processor, cause the processor to query a database for drug pharmaco-kinetics data and disease risk data.

19. The non-transitory computer readable medium of claim 18, further comprising instructions, that when read by the processor, cause the processor to generate the knowledge pre-trained embeddings based on the drug pharmaco-kinetics data and the disease risk data.

20. The non-transitory computer readable medium of claim 17, further comprising instructions, that when read by the processor, cause the processor to train a deep neural network y=f_{θ}(M,D,C) to predict the CT outcome based on model parameters θ.
